(19) United States
(12) Patent Application Publication
(10) Pub. No.: US 2024/0261509 A1
(43) Pub. Date: Aug. 8, 2024

(54) DRUG DELIVERY DEVICES, FINGER-GRIP ELEMENTS, AND RELATED METHODS

(71) Applicant: AMGEN INC., Thousand Oaks, CA (US)

(72) Inventors: Steve Sanchez, Thousand Oaks, CA (US); Scott Hanchar, Ventura, CA (US); Calvin Clark, Stow, MA (US)

(21) Appl. No.: 18/564,805

(22) PCT Filed: Jun. 2, 2022
(86) PCT No.: PCT/US22/31947

§ 371 Date: Nov. 28, 2023

Related U.S. Application Data
(60) Provisional application No. 63/208,322, filed on Jun. 8, 2021.

(51) Int. Cl. A61M 5/31; A61M 5/315

(52) U.S. Cl. CPC A61M 5/3137; A61M 5/31515; A61M 2005/3139; A61M 2005/3142; A61M 2205/586; A61M 2207/10

(57) ABSTRACT

Drug delivery devices, finger-grip elements for use with syringes, and related methods are disclosed. A drug delivery device may include a syringe and a finger-grip element. The syringe may include a barrel, a barrel flange extending radially outwardly with respect to a longitudinal axis of the barrel, and a plunger moveably disposed within the barrel. The finger-grip element may be coupled with the barrel flange. The finger-grip element may have a first distally facing surface having a first contour configured to: inhibit or prevent at least a first finger of a user from moving in a radially outward direction with respect to the longitudinal axis during use of the syringe to deliver a drug, and/or exert a first reaction force on at least the first finger of the user in a direction parallel or substantially parallel to the longitudinal axis during use of the syringe to deliver the drug.

### CROSS-REFERENCE TO RELATED APPLICATION

Priority is claimed to U.S. Provisional Patent Application No. 64/208,322, filed Jun. 8, 2021, the entire contents of which are hereby incorporated by reference herein.

### FIELD OF DISCLOSURE

The present disclosure generally relates to drug delivery devices and, more particularly, features that assist a user in handling and/or operating such devices.

### BACKGROUND

Drug delivery devices are used for administering drugs to patients. Certain drug delivery devices including syringes can be manually operated by a user to inject a drug into a patient. A syringe typically includes a barrel containing a drug and a plunger moveable with respect to the barrel to expel the drug via a needle or other fluid path member into the patient. Manually operating a syringe typically involves a user gripping the barrel between two fingers while pressing a plunger rod with the thumb of the same hand to move the plunger through the barrel, thereby discharging the drug.

Generally it is desirable for all or substantially all of the user's applied force to be transferred into motion of the plunger. Any inefficiency in transferring the applied force to the plunger can result in a deficit requiring the user to apply extra force to expel the drug. This burdens the user and in certain situations may be unacceptable, for example, where the drug is viscous and thus requires the application of significant force even under ideal conditions. Additionally, in applying extra force to expel the drug, the user's ability to hold the syringe steady with respect to the patient may be decreased. Any sudden movement of a needle of the syringe with respect to the patient during the injection, which might occur inadvertently as a result of the user applying extra force to move the plunger, has the potential to cause pain or discomfort to the patient.

The present disclosure sets forth devices and related methods embodying advantageous alternatives to existing drug delivery devices, syringes, add-on or accessory components for syringes, and related methods, and that may address one or more of the challenges or needs mentioned herein, as well as provide other benefits and advantages.

### SUMMARY

One aspect of the present disclosure provides a drug delivery device including a syringe and a finger-grip element. The syringe may include barrel, a barrel flange, and a plunger. The barrel may include a proximal end, a distal end, and a longitudinal axis. The barrel flange may be disposed at the proximal end of the barrel and extend radially outwardly with respect to the longitudinal axis. The plunger may be at least partially disposed within the barrel and moveable along the longitudinal axis. The finger-grip element may be coupled with the barrel flange and include a first distally facing surface having a first contour. The first contour may be configured to: inhibit or prevent at least a first finger of a user from moving in a radially outward direction with respect to the longitudinal axis during use of the syringe to deliver a drug, and/or exert a first reaction force on at least the first finger of the user in a direction parallel or substantially parallel to the longitudinal axis during use of the syringe to deliver the drug.

The first distally facing surface of the finger-grip element may include an outer portion and an inner portion, the inner portion being closer than the outer portion to the longitudinal axis. At least a portion of the outer portion may be distal to at least a portion of the inner portion.

The finger-grip element may include a second distally facing surface having a second contour. The second contour may be configured to: inhibit or prevent at least a second finger of a user from moving in a radially outward direction with respect to the longitudinal axis during use of the syringe to deliver the drug, and/or exert a second reaction force on at least the second finger of the user in a direction parallel or substantially parallel to the longitudinal axis during use of the syringe to deliver the drug.

The syringe may include a plunger rod coupled with the plunger. The finger-grip element may at least partially surround the plunger rod. A threaded exterior surface of the plunger rod may engage a threaded interior surface of the plunger to couple the plunger rod with the plunger. The plunger rod may include a plunger rod flange proximal to the threaded exterior surface. The plunger rod flange may extend radially outwardly with respect to the longitudinal axis. At least an inner portion of the distally facing surface of the plunger rod flange may contact a proximally facing exterior surface of the plunger. At least an outer portion of the distally facing surface of the plunger rod flange may be spaced from the proximally facing exterior surface of the plunger by a gap. A distal end of the plunger rod may be disposed at least partially within a recess formed in the plunger such that a distally facing end surface of the distal end of the plunger rod contacts a proximally facing interior surface of the plunger. Furthermore, the plunger rod may have a first axial length between the distally facing end surface of the distal end of the plunger rod and the distally facing surface of the plunger rod flange, and the plunger may have a second axial length between the proximally facing interior surface of the plunger and the proximally facing exterior surface of the plunger. The first axial length may be larger than the second axial length.

Another aspect of the present disclosure provides a finger-grip element for use with a syringe. The finger-grip element may include a proximal end and a distal end disposed at respective axial positions along a longitudinal axis. The finger-grip element may further include an opening configured to receive at least a portion of the syringe. The finger-grip element may additionally include a first distally facing surface having a first contour. The first contour may be configured to: (a) inhibit or prevent at least a first finger of a user from moving in a radially outward direction with respect to the longitudinal axis during use of the syringe to deliver a drug, and/or (b) exert a first reaction force on at least the first finger of the user in a direction parallel or substantially parallel to the longitudinal axis during use of the syringe to deliver the drug.

A further aspect of the present disclosure provides a method of making a finger-grip element for a syringe. The finger-grip element may have a length LFG1 corresponding to a distance between a longitudinal axis of a barrel of the syringe and a distally facing surface of the finger-grip element for contacting a finger of a user during use of the syringe to deliver a drug. The method may include selecting the length LFG1 for the finger-grip element such that M is equal to or approximately equal to zero in a mathematical formula wherein: M=(FFG1)(0.5)(LFG1)+((T)(cos(θ))+LB+LPR+(LF1)(Tan(θ)))(FPR)(Tan(30)). In this mathematical formula: FFG1 may correspond to a force applied to the distally directed surface of the finger-grip element by the finger of the user during use of the syringe to deliver the drug, T may correspond to a distance between a wrist of the user and a proximal end of a plunger rod of the syringe, LB may correspond to a length of barrel of the syringe, LPR may correspond to a length of the plunger rod of the syringe, LF1 may correspond to a length of the finger of the user, θ may correspond to an angle between the finger of the user and a thumb of the user during use of the syringe to deliver the drug, FPR may correspond to a force applied to the plunger rod by the thumb of the user during use of the syringe to deliver the drug, and/or M may correspond to a moment at or near a distal end of a needle of the syringe.

An additional aspect of the present disclosure provides a drug delivery device including a syringe and a finger-grip element. The syringe may include barrel, a barrel flange, and a plunger. The barrel may include a proximal end, a distal end, and a longitudinal axis. The barrel flange may be disposed at the proximal end of the barrel and extend radially outwardly with respect to the longitudinal axis. The plunger may be at least partially disposed within the barrel and moveable along the longitudinal axis. The finger-grip element may be coupled with the barrel flange and include a distally facing surface. During use of the syringe to deliver a drug, the finger-grip element may be configured to allow at least one finger of a user to contact concurrently the distally facing surface of the finger-grip element and the barrel of the syringe.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings is necessarily to scale.

FIG. 1 is a perspective view of an exemplary drug delivery device in accordance with various embodiments.

FIG. 2 is a side view of the drug delivery device shown in FIG. 1.

FIG. 3 is a side view of a syringe of the drug delivery device depicted in FIG. 1.

FIG. 4 is a perspective view of the finger-grip element of the drug delivery device shown in FIG. 1.

FIG. 5 is another perspective view of the finger-grip element of the drug delivery device shown in FIG. 1.

FIG. 6 is a side view of another exemplary drug delivery device in accordance with various embodiments.

FIG. 7 is a perspective view of the drug delivery device illustrated in FIG. 6.

FIG. 8 is a perspective view of the finger-grip element of the drug delivery device shown in FIGS. 6 and 7.

FIG. 9 is a perspective view of another exemplary drug delivery device in accordance with various embodiments.

FIG. 10 is a side view of another exemplary drug delivery device in accordance with various embodiments.

FIG. 11 is a side view of another exemplary drug delivery device in accordance with various embodiments.

FIG. 12 is a side view of another exemplary drug delivery device in accordance with various embodiments.

FIG. 13 is a perspective view of another exemplary drug delivery device in accordance with various embodiments.

FIG. 14 is a side view of the drug delivery device shown in FIG. 13.

FIG. 15 is a cross-sectional view taken along longitudinal axis A of the drug delivery device shown in FIG. 14.

FIG. 16 is an enlarged cross-sectional view of a plunger and a plunger rod of the drug delivery device shown in FIG. 15.

FIG. 17 is a perspective view of the plunger rod of the drug delivery device shown in FIGS. 13-16.

FIG. 18 is an enlarged view of the distal end of the plunger rod shown in FIG. 17.

FIG. 19 is a perspective view of another exemplary finger-grip element in accordance with various embodiments.

FIG. 20 is a side view of the finger-grip element in FIG. 19.

FIG. 21 is a perspective view of another exemplary finger-grip element in accordance with various embodiments.

FIG. 22 is a side view of the finger-grip element in FIG. 21.

FIG. 23 is a perspective view of the finger-grip element of FIG. 21 being used by a user in conjunction with a syringe.

### DETAILED DESCRIPTION

The present disclosure generally relates to drug delivery devices operable by a user for administering a drug to a patient, or in a scenario where the user is the patient, self-administering a drug, as well as subassemblies and components for drug delivery devices and methods of making such devices, subassemblies, and components. A drug delivery device according to the present disclosure may include, for example, a syringe and a finger-grip element having one or more contact surfaces for a user to press with one or more fingers while operating the syringe to deliver a drug to, for example, a patient. As described below, the finger-grip element may facilitate an efficient transfer of a user's applied force into axial motion of a plunger of the syringe, improve the user's ability to grip a syringe as compared to a syringe lacking the presently-disclosed finger-grip element, provide a user with mechanical leverage in operating the syringe to expel the drug, and/or provide other benefits and advantages. Additional features are disclosed, which, when used in conjunction with or independently of the presently-disclosed finger-grip element, can improve the operation of the syringe, including, for example, reducing an amount force required to push a plunger through a barrel of the syringe. These and other advantages and benefits will be apparent to one of ordinary skill in the art reviewing the present disclosure.

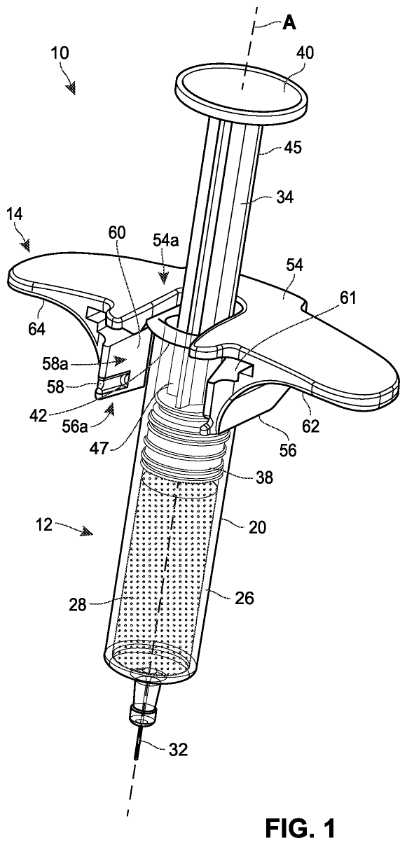

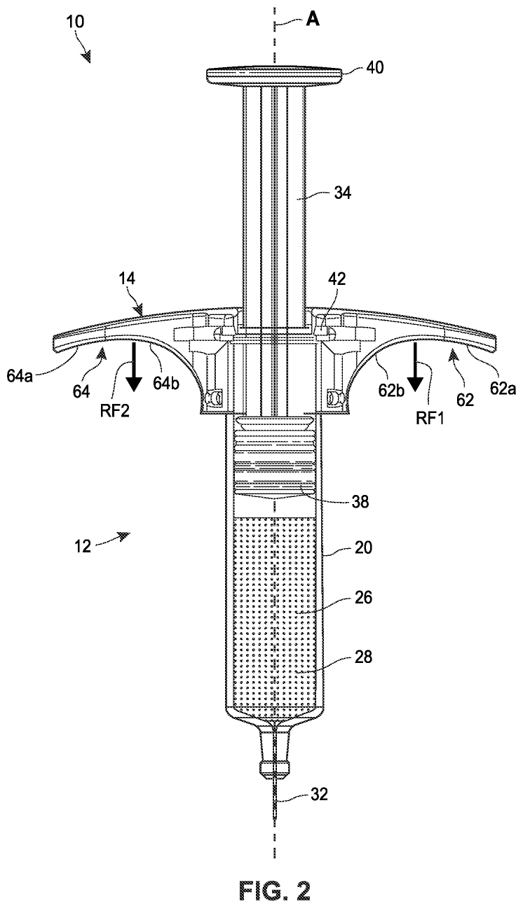

FIGS. 1 and 2 illustrate several views of an embodiment of a drug delivery device 10 for delivering a drug, which may also be referred to herein as a medicament or drug product, to, for example, a patient. The drug may be, but is not limited to, various biologics such as peptides, peptibodies, and/or antibodies. The drug may be in a fluid or liquid form, gelatin form, and/or powdered form, although the present disclosure is not limited to a particular form or state of the drug. The drug delivery device 10 illustrated in FIGS. 1 and 2 may be in its final assembled form and/or in a pre-delivery or storage state prior to use or operation by a user.

The drug delivery device 10 may include a syringe 12 and a finger-grip element 14 coupled with (e.g., removably coupled with) an exterior of the syringe 12. The finger-grip element 14 may be an accessory or add-on component that initially is separate from the syringe 12 and subsequently coupled with the syringe 12 prior to operating the syringe 12 to deliver the drug. As an example, a user of the syringe 12 may be responsible for coupling the finger-grip element 14 with the syringe 12. The user of the syringe 12 may be a formally trained healthcare professional (e.g., a doctor, a nurse, a nurse practitioner, etc.), a caregiver, a patient, and/or any other individual and/or a machine. In other embodiments, an individual and/or entity different from the user may be responsible for coupling the finger-grip element 14 with the syringe 12, including, for example, pharmacist, a manufacturer, a drug manufacturer, a medical device manufacturer, a supplier, and/or a medical device supplier. A benefit of configuring the finger-grip element 14 as an accessory or add-on component is that one can improve the usability and/or ergonomics of the syringe 12 without necessarily having to modify an existing design and/or existing method of manufacturing the syringe 12, which in certain cases may be standardized and thus expensive and/or impractical to change. In alternative embodiments, the finger-grip element 14 may be integrally formed with the syringe 12 such that at least a portion of the finger-grip element 14 and at least a portion of syringe 12 form a single, unitary structure and/or are permanently attached to one another.

The syringe 12 may include a barrel 20 (which is illustrated as being transparent in the figures) having a proximal end 22, a distal end 24, and a longitudinal axis A. The proximal end 22 and the distal end 24 of the barrel 20 may be arranged at respective axial positions along the longitudinal axis A, as seen in FIGS. 1-3. The barrel 20 may have a generally cylindrical wall 26 defining, partially or entirely, an internal bore or reservoir 28 of the syringe 12. At least the cylindrical wall 26 of the barrel 20 may be made of a rigid or semi-rigid material including, e.g., glass, plastic (e.g., polypropylene), and/or any other suitable material or combination of materials. At least a portion of or the entirety of the barrel 20 may have cross-sectional shape (e.g., in a plane perpendicular to the longitudinal axis A) that is generally circular or any other suitable shape.

The reservoir 28 of the syringe 12 may be filled partially or entirely with a drug. In some embodiments, the syringe 12 may be pre-filled with a drug and shipped, e.g., by a manufacturer, to a location where the syringe 12 is combined with the finger-grip element 14. In other embodiments, the reservoir 28 of syringe 12 may be provided to the user empty and then the user fills the reservoir 28 at the point-of-care, for example, by transferring the drug from a vial or other drug storage container into the syringe 12 immediately or substantially immediately before using the syringe 12 to perform an injection.

In some embodiments, a volume of the drug stored in the reservoir 28 of the syringe 12 may be equal to 1 mL, or equal to approximately (e.g., ±10%) 1 mL, or equal to 2.5 mL, or equal to approximately (e.g., ±10%) 2.5 mL, or equal to 3 mL, or equal to approximately (e.g., ±10%) 3 mL, or less than or equal to approximately (e.g., ±10%) 1 mL, or less than or equal to approximately (e.g., ±10%) 2 mL, or less than or equal to approximately (e.g., ±10%) 3 mL, or less than or equal to approximately (e.g., ±10%) 4 mL, or less than approximately (e.g., ±10%) 5 mL, or less than or equal to approximately (e.g., ±10%) 10 mL, or within a range between approximately (e.g., 10%) 1-10 mL, or within a range between approximately (e.g., ±10%) 1-5 mL, or within a range between approximately (e.g., ±10%) 1-4 mL, or within a range between approximately (e.g., ±10%) 1-3 mL, or within a range between approximately (e.g., ±10%) 1-2.5 mL.

The proximal end 22 of the barrel 20 may include a proximal axial opening 22a through which a plunger rod 34 extends. The distal end 24 of the barrel 20 may include a distal axial opening 24b configured to provide fluid communication with the drug in the reservoir 28 of the syringe 12. In some embodiments, a needle 32 may be staked or otherwise coupled with the distal end 24 of the barrel 20 and/or in fluid communication with the reservoir 28 via the distal axial opening 24b. The needle 32 may fixed (e.g., adhered and/or staked) to the wall 26 of the barrel 20 such that the needle 32 cannot move with respect to the wall 26 of the barrel 20. A distal end 33 of the needle 32 may include a sharpened tip or other pointed geometry allowing the distal end 33 of the needle 32 to pierce and/or penetrate through a patient's skin, subcutaneous tissue, and/or other tissue. The needle 32 may be hollow and/or include an axial passage that is parallel to and/or coaxial with the longitudinal axis A of the barrel 20. One or more openings may be formed in the distal end 33 of the needle 32 to allow drug to flow out of the needle 32 into the patient during use of the syringe 12. The needle 32 may be made of metal and/or any other suitably rigid material. Prior to use, the distal end 33 of the needle 32 may be covered with a removable sterile barrier such as, for example, a rigid needle shield (RNS) or a non-rigid needle shield (nRNS). The removable sterile barrier may be configured to protect the needle 32 from contaminants in an external or ambient environment prior to use of the syringe 12. The user may be required to remove the removable sterile barrier from the needle 32 prior to using the syringe 12. In alternative embodiments, the needle 32 may be omitted or at least not directly attached to the wall 26 of the barrel 20. In such alternative embodiments, the distal end 24 of the barrel 20 may be coupled with and/or form a nozzle or other fluid path member including, for example, a Luer Lock fitting.

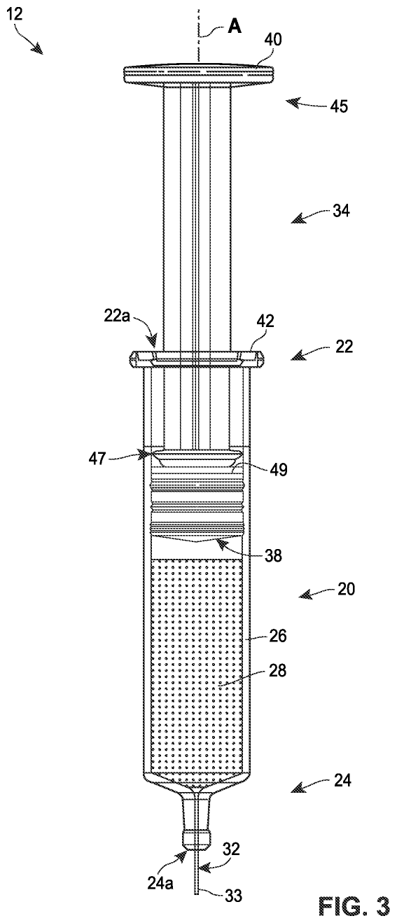

As best seen in FIG. 3, a barrel flange 42 may be disposed at the proximal end 22 of the barrel 20. The barrel flange 42 may be integrally formed with the wall 26 of the barrel 20 such that the barrel flange 42 and the wall 26 define a single, unitary structure; or, alternatively, the barrel flange 42 and the wall 26 of the barrel 20 may be separate structures that are rigidly coupled with each other. The barrel flange 42 may generally extend in a radially outwardly direction with respect to the longitudinal axis A of the barrel 20. The barrel flange 42 may extend completely or partially around the barrel 20. In some embodiments, two or more distinct barrel flanges may extend radially outwardly from respective portions of the wall 26 of the barrel 20. The barrel flange 42 may be included by a syringe manufacturer, for example, to prevent the syringe 12 from inadvertently rolling across a flat surface when lying on its side. Due to its relatively small overhang or radial width, at least some users may find it challenging or impossible to use solely the barrel flange 42 as a finger grip during use of the syringe 12 to deliver a drug.

The syringe 12 may additionally include a plunger 38 (also referred to in some contexts as a “stopper”). The plunger 38 may be moveably disposed in the barrel 20 such that it can move at least in a distal direction along the longitudinal axis A from an initial position near the proximal end 22 of the barrel 22 to at least an end-of-dose or end-of-delivery position near the distal end 24 of the barrel 22. Proximal movement of the plunger 38 along the longitudinal axis A may also be possible in at least some embodiments. The plunger 38 may be constructed of an elastic material such as rubber or any other suitable material. The plunger 38 may slidably and/or sealingly contact an interior surface of the wall 26 of the barrel 20 such that, for example, the drug in the reservoir 28 is prevented or inhibited from leaking past the plunger 38 when the plunger 38 moves in the distal direction. As an example, the plunger 38 may form a fluid-tight seal with the interior surface of the wall 26 of the barrel 20. Distal movement of the plunger 38 may expel the drug from the reservoir 28 of the syringe 12 into and out through the needle 32.

The plunger rod 34 may include a proximal end 45 and a distal end 47. The distal end 47 of the plunger rod 34 may be coupled with (e.g., removably coupled with) a proximal end 49 of the plunger 38. In some embodiments, this coupling may be achieved via one or more threaded surfaces, as described below in more detail. Generally, the plunger rod 34 may be configured to allow a user to manually or semi-manually move the plunger 38 in the distal and/or proximal direction along the longitudinal axis A. As an example, the plunger rod 34 may have a longitudinal axis that is parallel to and/or coaxial with the longitudinal axis A of the barrel 20. As a more specific example, the proximal end 45 of the plunger rod 34 may include a thumb rest 40 allowing a user to press his or her thumb against the plunger rod 34 to push the plunger rod 34 and thereby the plunger 38 in the distal direction along the longitudinal axis A to expel the drug from the reservoir 28 through the needle 32 and into, for example, the patient's tissue. To draw drug from a vial or other drug storage container into the reservoir 28 of the syringe 12, a user may pull up on the thumb rest 40, thereby moving the plunger 38 in a proximal direction along the longitudinal axis A. This action may create a vacuum capable of suctioning the drug into the reservoir 28 via, for example, the needle 32.

Referring to FIGS. 1, 2, 4, and 5, the finger-grip element 14 will now be described in more detail. Generally the finger-grip element 14 may be configured to facilitate a user's grip of the barrel 20 of the syringe 12 during, for example, use of the syringe 12 to deliver the drug to, for example, a patient. The finger-grip element 14 may increase the surface area available for one or more of the user's fingers to press against while, for example, the user presses his or her thumb against the proximal end 45 of plunger rod 34. As an example, the user may press his or her index finger and/or middle finger against the finger-grip element 14 to apply one or more proximally directed forces to the finger-grip element 14 while simultaneously pressing his or her thumb of the same hand against the thumb rest 40 to apply a distally directed force to the plunger rod 34. The finger-grip element 14 may be considered to take the form of a flange extender in the sense that it generally serves to extend the radial length of the barrel flange 42.

The finger-grip element 14 may have a proximal end 54 and a distal end 56. One or more axial openings may be formed in the finger-grip element 14 including, for example, a proximal axial opening 54a formed in the proximal end 54 of the finger-grip element 14 and a distal axial opening 56a formed in the distal end 56 of the finger-grip element 14. When the finger-grip element 14 is coupled with the syringe 12, the longitudinal axis A of the barrel 20 of the syringe 12 may pass through (e.g., pass centrally through) the proximal axial opening 54a and/or the distal axial opening 56a. Additionally, in some embodiments, one or more side openings may be formed in the finger-grip element 14 including, for example, a side opening 58a formed in a lateral side 58 of the finger-grip element 14. When viewed along the longitudinal axis A, the finger-grip element 14 may be generally C-shaped or U-shape, at least in part because of the side opening 58a. In other embodiments, the finger-grip element 14 may have no side openings such that the finger-grip element 14 completely surrounds the barrel 20 and/or plunger rod 34.

An axial passage 60 may extend through the finger-grip element 14 and may be configured to receive at least a portion of the barrel 20, the barrel flange 42, and/or the plunger rod 34. The proximal axial opening 54a, the distal axial opening 56a, and/or the side opening 58a may open into or otherwise communicate with the axial passage 60. As an example, the side opening 58a may be configured to permit the barrel 20, the barrel flange 42, and/or the plunger rod 34 to be inserted laterally into the axial passage 60 of the finger-grip element 14. As a more specific example, laterally inserting the barrel 20, the barrel flange 42, and/or the plunger rod 34 into the axial passage 60 may involve moving the barrel 20, the barrel flange 42, and/or the plunger rod 34 in a direction that is perpendicular to or otherwise transverse to the longitudinal axis A such that a lateral side of the barrel 20, the barrel flange 42, and/or the plunger rod 34 is initially received within the axial passage 60. As a further example, laterally inserting the barrel 20, the barrel flange 42, and/or the plunger rod 34 into the axial passage 60 may involve inserting the barrel 20, the barrel flange 42, and/or the plunger rod 34 in a non-axial direction or radial direction into the axial passage 60.

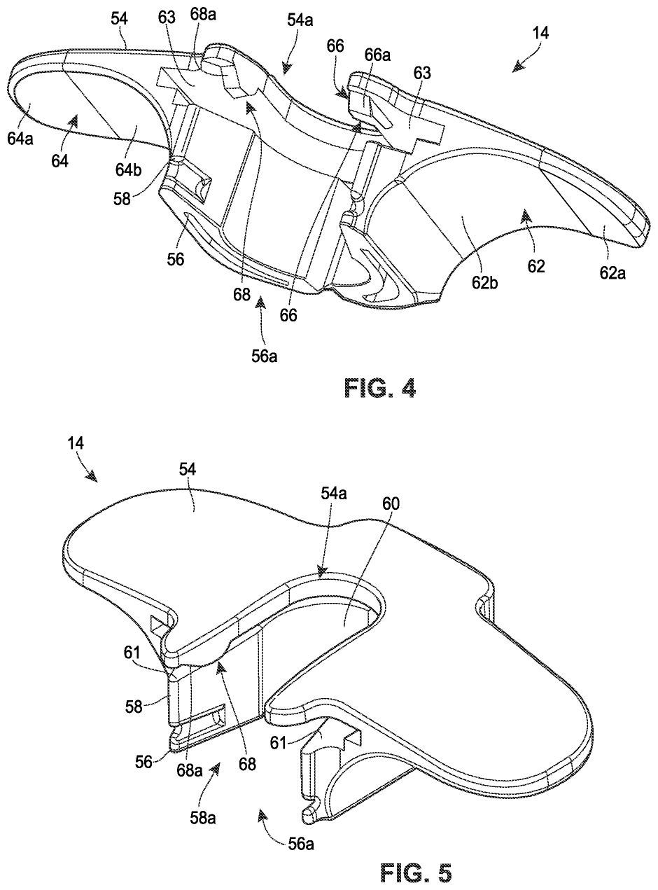

Coupling the finger-grip element 14 to the syringe 12 may involve coupling the finger-grip element 14 with, for example, the barrel flange 42 of the syringe 12. The finger-grip element 14 may be configured to removably or permanently couple with the barrel flange 42. In the embodiment illustrated in FIGS. 1-5, the finger-grip element 14 is configured to form a snap-fit coupling with the barrel flange 42. As an example, a recess 60 (e.g., a cavity, slot, depression, channel, interior space, etc.) may be formed at least in an inner wall of the finger-grip element 14 and may be configured to receive the barrel flange 42 when the barrel 22 is inserted into the finger-grip element 14 via the side opening 58a. The recess 60 may communicate with side opening 58a such that the barrel flange 20 can be inserted into the recess 60 via the side opening 58a. Furthermore, the recess 60 may be bordered in the axial direction by a proximally facing interior surface 61 of the finger-grip element 14 and a distally facing interior surface 63 of the finger-grip element 63, as seen in FIGS. 4 and 5. The snap-fit coupling may be facilitated by a first locking tab 66 and/or a second locking tab 68. The first locking tab 66 and/or the second locking tab 68 may extend in the distal direction away from the distally facing interior surface 63 and/or may be disposed adjacent to the side opening 58a. As an example, the first locking tab 66 and/or the second locking tab 68 may be configured to permit the barrel flange 42 to be inserted into the recess 60 in a first radial direction and/or inhibit or prevent removal of the barrel flange 42 from the recess in a second radial direction (e.g., in a radial direction opposite to the first radial direction). As a more specific example, the first locking tab 66 and/or the second locking tab 68 may be configured such that the force needed to insert the barrel flange 42 into the recess 60 in the first radial direction is substantially less than the force needed to remove the barrel flange 42 from the recess 60 in the second radial direction. As an even more specific example, the first locking tab 66 and the second locking tab 68 may have, respectively, a lead-in surface 66a and a lead-in surface 68a, each of which may be angled with respect to the radial or horizontal direction, for example, at an angle less than 90 degrees, or at an angle less than or equal to approximately (e.g., ±10%) 75 degrees, or at an angle less than or equal to approximately (e.g., ±10%) 60 degrees, or at an angle less than or equal to approximately (e.g., ±10%) 45 degrees, or at an angle less than or equal to approximately (e.g., ±10%) 30 degrees. Inserting the barrel flange 42 into the recess 60 may involve sliding the barrel flange 42 along and/or against the lead-in surfaces 66a and 68a as well as along and/or against the proximally facing interior surface 61 of the finger-grip element 14 such that at least a portion of the proximal end 54 of the finger-grip element 14 and at least a portion of the distal end 56 of the finger-grip element 14 deflect (e.g., elastically deflect) away from each other. Once the barrel flange 42 has cleared the locking tabs 66 and 68, the proximal end 54 and/or the distal end 56 of the finger-grip element 14 may elastically return or snap back to its original shape, thereby temporarily or permanently locking the barrel flange 42 within the recess 60 of the finger-grip element 14. In some embodiments, the snap-fit aspect of the finger-grip element 14 and/or other aspect(s) of the finger-grip element 14 may be the same as or similar to those described in International Patent Application Publication No. WO 2017/039786, the entire contents of which are hereby incorporated by reference.

The finger-grip element 14 may include one or more generally distally facing exterior surfaces for contacting one or more fingers of the user during use of the syringe 12 to deliver a drug to, for example, a patient. As an example, the user may press his or her finger(s) against the distally facing exterior surface(s), thereby applying generally proximally directed force(s) to the distally facing exterior surface(s) during use of the syringe 12 to deliver the drug. Generally, the distally facing exterior surface(s) of the finger-grip element 14 may be configured such that the mechanics of the force(s) applied by the user to the finger-grip element 14 facilitate an efficient transfer force from the plunger rod 34 into motion of the plunger 38 to expel the drug from the syringe 12 (e.g., motion of the plunger 38 in the distal direction along the longitudinal axis A). As an example, the distally facing exterior surface(s) of the finger-grip element 14 may be configured to prevent or inhibit the user's finger(s) from slipping in the radial and/or axial direction(s), thereby maintaining the finger(s) at respective mechanically desirable location(s) (e.g., a desired radial distance measured from the longitudinal axis A). Additionally or alternatively, the distally facing exterior surface(s) of the finger-grip element 14 may be configured to apply reaction force(s) to the user's finger(s) in a mechanically desirable direction (e.g., in a direction parallel or substantially parallel to the longitudinal axis A). In facilitating an efficient transfer of user applied force(s) into motion of the plunger 38, the finger-grip element 14 may reduce or minimize force loss(es) as compared to conventional syringes and/or make the syringe 12 easier and/or less cumbersome to operate for the user.

As seen in FIGS. 2 and 4, the finger-grip element 14 may have a first distally facing exterior surface 62 and a second distally facing exterior surface 64. The first distally facing exterior surface 62 may have a first contour, and the second distally facing exterior surface 64 may have a second contour. The first contour of the first distally facing exterior surface 62 may be the same as or similar to, or different from, the second contour of the second distally facing exterior surface 64. As an example, at least the first contour of the first distally facing exterior surface 62 and at least the second contour of the second distally facing exterior surface 64 may be symmetric or substantially symmetric with each other across the longitudinal axis A.

The first contour of the first distally facing exterior surface 62 may be configured to: (a) inhibit or prevent at least a first finger (e.g., an index finger or a middle finger) of the user from moving in a radially outward and/or radially inward direction with respect to the longitudinal axis A of the barrel 20 during use of the syringe 12 to deliver the drug; and/or (b) exert a first reaction force RF1 on at least the first finger of the user in a direction parallel or substantially parallel to the longitudinal axis A of the barrel 20 during use of the syringe 12 to deliver the drug. So configured, the first contour of the first distally facing exterior surface 62 may, for example, assist in positioning and/or maintaining the first finger of the user at predetermined position with respect to the barrel 20 during use of the syringe 12 to deliver the drug (e.g., at a position that is spaced apart from the longitudinal axis A of the barrel 20 by a radial distance providing a desirable mechanical effect) and/or limit or eliminate mechanically undesirable moment(s) or torque(s) potentially resulting from user applied force(s) during use of the syringe 12 to deliver the drug. As an example, a portion of or the entirety of the first contour of the first distally facing exterior surface 62 may have a concave curvature configured to: inhibit or prevent at least a first finger of the user from moving in a radially outward and/or radially inward direction with respect to the longitudinal axis A of the barrel 20 during use of the syringe 12 to deliver the drug; and/or exert the first reaction force RF1 on at least the first finger of the user in a direction parallel or substantially parallel to the longitudinal axis A of the barrel 20 during use of the syringe 12 to deliver the drug. As a more specific example, the concave curvature may be configured such that the proximal-most portion of the first distally facing exterior surface 62 has a radius of curvature that corresponds to or complements a radius of curvature of at least a portion of the index, middle, or other finger of an average adult or other user. As an even more specific example, at least a portion of the concave curvature may have a radius of curvature that varies along the first distally facing exterior surface 62, including, for example, a radius of curvature that becomes increasingly larger moving generally in a radial direction away from the longitudinal axis A. In addition to or as an alternative to the concave curvature, the first distally facing exterior surface 62 may have an outer portion that is flared in a distal or downward direction, as best seen in FIG. 2. As an example, the first distally facing exterior surface 62 may have an outer portion 62a and an inner portion 62b, such that the inner portion 62b is closer than the outer portion 62a to the longitudinal axis A, and such that a portion of or the entirety of the outer portion 62a is distal to or below a portion of or the entirety of the inner portion 62b. As a more specific example, a radially outermost portion of the first distally facing exterior surface 62 may be distal to or below a portion of the first distally facing exterior surface 62 that contacts and/or presses against a central portion of the first finger of the user during use of the syringe 12 to deliver the drug. The distally directed flare of the outer portion 62a of the first distally facing exterior surface 62 may inhibit or prevent the first finger of the user from inadvertently slipping or moving along the first distally facing exterior surface 62 in a radially outward direction during use of the syringe 12 to deliver the drug. As a consequence, the first finger of the user may be restrained to a predetermined or desired position along the first distally facing exterior surface 62 where the force applied by the first finger has mechanically advantageous effect(s).

The second contour of the second distally facing exterior surface 64 may be configured to: (a) inhibit or prevent at least a second finger (e.g., an index finger or a middle finger) of the user from moving in a radially outward and/or radially inward direction with respect to the longitudinal axis A of the barrel 20 during use of the syringe 12 to deliver the drug; and/or (b) exert a second reaction force RF2 on at least the second finger of the user in a direction parallel or substantially parallel to the longitudinal axis A of the barrel 20 during use of the syringe 12 to deliver the drug. So configured, the second contour of the second distally facing exterior surface 64 may, for example, assist in positioning and/or maintaining the second finger of the user at predetermined position with respect to the barrel 20 during use of the syringe 12 to deliver the drug (e.g., at a position that is spaced apart from the longitudinal axis A of the barrel 20 by a radial distance providing a desirable mechanical effect) and/or limit or eliminate mechanically undesirable moment(s) or torque(s) potentially resulting from user applied force(s) during use of the syringe 12 to deliver the drug. As an example, a portion of or the entirety of the second contour of the second distally facing exterior surface 64 may have a concave curvature configured to: inhibit or prevent at least a second finger of the user from moving in a radially outward and/or radially inward direction with respect to the longitudinal axis A of the barrel 20 during use of the syringe 12 to deliver the drug; and/or exert the second reaction force RF2 on at least the second finger of the user in a direction parallel or substantially parallel to the longitudinal axis A of the barrel 20 during use of the syringe 12 to deliver the drug. As a more specific example, the concave curvature may be configured such that the proximal-most portion of the second distally facing exterior surface 64 has a radius of curvature that corresponds to or complements a radius of curvature of at least a portion of the index, middle, or other finger of an average adult or other user. As an even more specific example, at least a portion of the concave curvature may have a radius of curvature that varies along the second distally facing exterior surface 64, including, for example, a radius of curvature that becomes increasingly larger moving generally in a radial direction away from the longitudinal axis A. In addition to or as an alternative to the concave curvature, the second distally facing exterior surface 64 may have an outer portion that is flared in a distal or downward direction, as best seen in FIG. 2. As an example, the second distally facing exterior surface 64 may have an outer portion 64a and an inner portion 64b, such that the inner portion 64b is closer than the outer portion 64a to the longitudinal axis A, and such that a portion of or the entirety of the outer portion 64a is distal to or below a portion of or the entirety of the inner portion 64b. As a more specific example, a radially outermost portion of the second distally facing exterior surface 64 may be distal to or below a portion of the second distally facing exterior surface 64 that contacts and/or presses against a central portion of the second finger of the user during user of the syringe 12 to deliver the drug. The distally directed flare of the outer portion 64a of the second distally facing exterior surface 64 may inhibit or prevent the second finger of the user from inadvertently slipping or moving along the second distally facing exterior surface 64 in a radially outward direction during use of the syringe 12 to deliver the drug. As such, the second finger of the user may be restrained to a predetermined or desired position along the second distally facing exterior surface 64 where the force applied by the second finger has mechanically advantageous effect(s).

In some embodiments, when the user presses his or her first finger against the first distally facing exterior surface 62, the reaction force RF1 may be the sole reaction force exerted by the first distally facing exterior surface 62 on the first finger of the user. For example, the first distally facing exterior surface 62 may exert no or de minimis (e.g., infinitesimally small) reaction force(s) in a radial direction with respect to the longitudinal axis A, such that any and/or all reaction force(s) exerted by the first distally facing exterior surface 62 on the first finger of the user are parallel or substantially parallel to the longitudinal axis A. Similarly, in some embodiments, when the user presses his or her first second against the second distally facing exterior surface 64, the reaction force RF2 may be the sole reaction force exerted by the second distally facing exterior surface 64 on the second finger of the user. For example, the second distally facing exterior surface 64 may exert no or de minimis (e.g., infinitesimally small) reaction force(s) in a radial direction with respect to the longitudinal axis A, such that any and/or all reaction force(s) exerted by the second distally facing exterior surface 64 on the second finger of the user are parallel or substantially parallel to the longitudinal axis A. In some embodiments, reaction force RF1 may be equal or approximately equal to reaction force RF2.

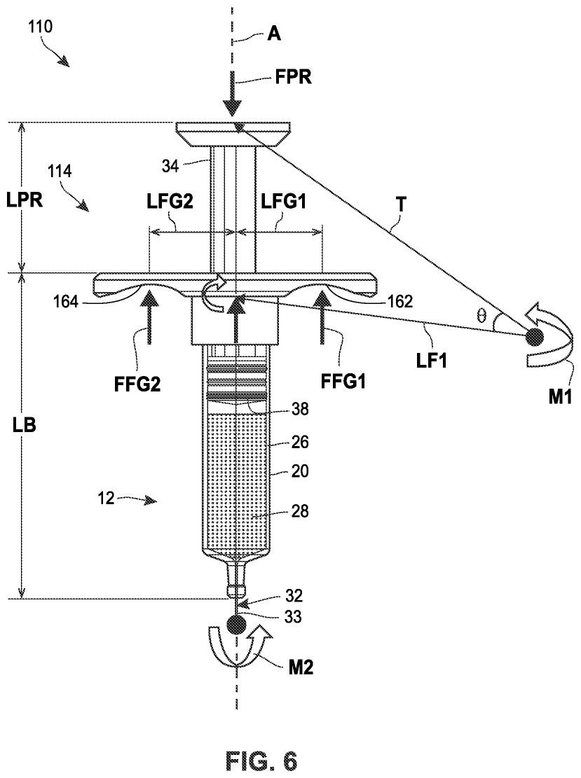

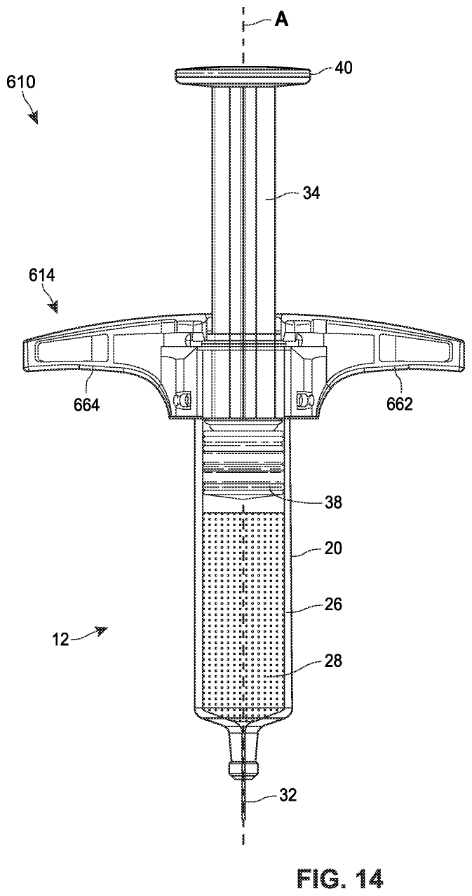

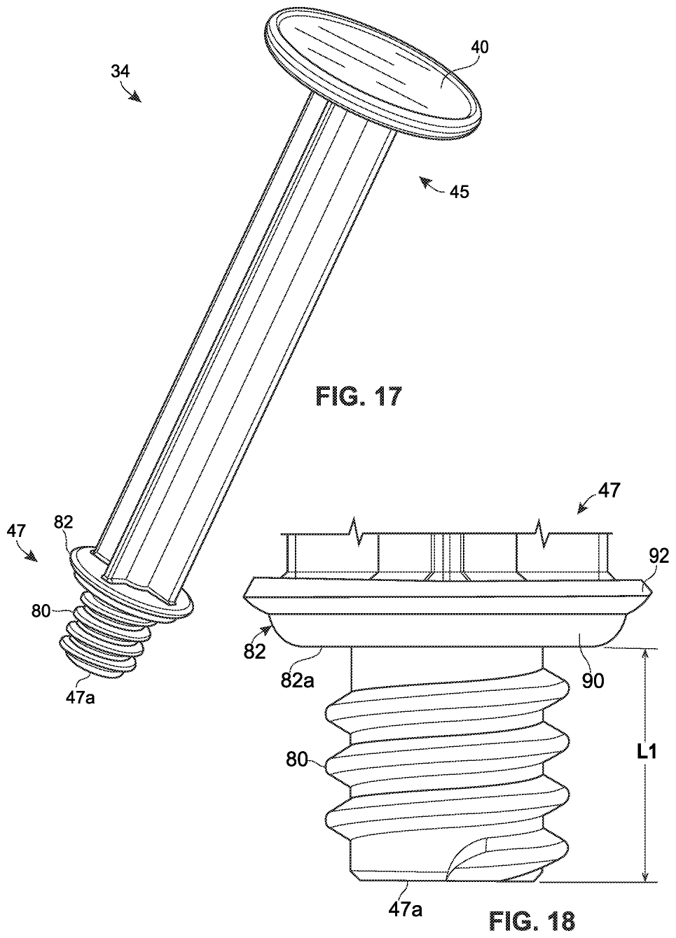

Turning to FIGS. 6-23, additional embodiments of a finger-grip element will now be described. Various features of the finger-grip elements illustrated in FIGS. 6-23 may be similar or identical in structure, configuration, and/or function to features of the finger-grip element 14 described above in conjunction with FIGS. 1-5. Such features are assigned with the same reference numeral as used in FIGS. 1-5, except incremented by 100 or a multiple thereof. A description of some of these features is abbreviated or eliminated in the interest of conciseness.

A method of making a finger-grip element, including, for example, the embodiment of the finger-grip element illustrated in FIGS. 1-5 and other embodiments of a finger-grip element described herein, will now be described with reference to FIG. 6.

Similar to the finger-grip element 14, the finger-grip element 114 may have a first distally facing exterior surface 162 and a second distally facing exterior surface 164 to be pressed by, respectively, a first finger (e.g., one of an index finger and a middle finger) and a second finger (e.g., the other one of the index finger and the middle finger) of a user during use of the syringe 12 to deliver (e.g., expel) a drug. As an example, to expel the drug from the syringe 12 the user may press his or her fingers against the first and second distally facing exterior surfaces 162 and 164 of the finger-grip element 114 in the proximal direction while simultaneously pressing his or her thumb (e.g., of the same hand as the fingers) against the thumb rest 40 of plunger rod 34 in the distal direction.

FIG. 6 depicts various dimensions of the syringe 12, finger-grip element 114 and user, various forces applied by the user during use of the syringe 12 to deliver a drug to a patient, and various moments resulting from the user applied forces.

More particularly with reference to FIG. 6, the finger-grip element 114 may have a first length LFG1 corresponding to a distance (e.g., a radial distance) between the longitudinal axis A of the barrel 20 of the syringe 12 and a portion (e.g., the proximal-most portion) of the first distally facing exterior surface 162 to be pressed by the first finger of the user during use of the syringe 12 to deliver the drug. The finger-grip element 114 may have a second length LFG2 corresponding to a distance (e.g., a radial distance) between the longitudinal axis A of the barrel 20 of the syringe 12 and a portion (e.g., the proximal-most portion) of the second distally facing exterior surface 164 to be pressed by the second finger of the user during use of the syringe 12 to deliver the drug.

The barrel 20 of the syringe 12 may have a length LB parallel to the longitudinal axis A. In some embodiments, the length LB of the barrel 20 may be measured between the proximal end 22 of the barrel 20 and the distal end of the barrel 20; whereas, in other embodiments, the length of the barrel 20 may be measured between the proximal end 22 of the barrel 20 and the distal end 33 of the needle 32.

The plunger rod 34 of the syringe 12 may have a length LPR parallel to the longitudinal axis A. In some embodiments, the length LPR may correspond to the length of the portion of the plunger rod 34 proximal to (thus outside of) the proximal end 22 of the barrel 20; whereas, in other embodiments, the length LPR may be measured between the proximal end 45 of the plunger rod 34 and the distal end 47 of the plunger 45.

Distance T in FIG. 6 corresponds to a distance between a wrist of the user (e.g., a center or central portion of a wrist of the user) and the proximal end 45 of plunger rod 34. As an example, the distance T may correspond to a distance between a center or central portion of a wrist of an average adult or other user and the thumb rest 40 of the plunger rod 34.

Length LF1 in FIG. 6 corresponds to a length of the first finger of the user. As an example, length LF1 may correspond to the length of the index of the index finger of the user, or the length of the middle finger, or the length of any other finger of the user used to press against the first distally facing exterior surface 162 of the finger-grip element 114 to effectuate drug delivery via the syringe 12.

Angle θ in FIG. 6 corresponds to an angle between the first finger of the user (e.g., the index or middle finger of the user) and the thumb of the same hand of the user during use of the syringe 12 to deliver the drug. As an example, the angle θ may correspond to the angle between the first finger of the user when the first finger is pressed against the first distally facing exterior surface 162 of the finger-grip element 114 and the thumb of the user when the thumb is pressed against the thumb rest 40 of the plunger rod at the start of drug delivery or at any time during drug delivery.

Force FFG1 in FIG. 6 corresponds to a force applied to the first distally directed surface 162 of the finger-grip element 114 by the first finger of the user. As an example, the force FFG1 may be applied in the proximal direction to the first distally directed surface 162. As a more specific example, the force FFG1 may be parallel or substantially parallel to the longitudinal axis A. Force FFG2 in FIG. 6 corresponds to a force applied to the second distally directed surface 164 of the finger-grip element 114 by the second finger of the user. As an example, the force FFG2 may be applied in the proximal direction to the second distally directed surface 164. As a more specific example, the force FFG2 may be parallel or substantially parallel to the longitudinal axis A. As an even more specific example, the force FFG2 may be parallel or substantially parallel to the force FFG1.

Force FPR in FIG. 6 corresponds to a force applied to the proximal end 45 of plunger rod 34 by the thumb or other part of the hand of the user. As an example, the force FPR may be applied in the distal direction to the thumb rest 40 of the plunger rod 34. As a more specific example, the force FPR may be parallel or substantially parallel to the longitudinal axis A. As an even more specific example, the force FPR may be perpendicular or substantially perpendicular to the force FFG1 and/or the force FFG2. According to some embodiments, FPR may be related to FFG1 and/or FFG1 so as to satisfy the following mathematical formulas, respectively, formula I and formula II:

FPR=(0.5)(FFG1)+(0.5)(FFG2)

FFG1=(2)(FPR−(0.5)(FFG2))

M1 in FIG. 6 corresponds to a moment at or near the wrist and/or hand of the user during use of the syringe 12 to deliver the drug. As an example, the moment M1 may be created at least in part by the user's first finger applying the force FFG1 to the finger-grip element 114 and/or the user's thumb applying the force FPR to the plunger rod 34. According to some embodiments, the moment M1 may be related to FFG1, LFG1, FPR, and/or T so as to satisfy the following mathematical formula III:

M1=(FFG1)(0.5)(LFG1)+(FPR)(Tan(30))(T)(cos(θ))

M2 in FIG. 6 corresponds to a moment created at or near the distal end 33 of the needle 32 and/or the tissue adjacent the distal end 33 of the needle 32 during use of the syringe 12 to deliver the drug. As an example, the moment M1 may be created at least in part by the user's first finger applying the force FFG1 to the finger-grip element 114 and/or the user's thumb applying the force FPR to the plunger rod 34. According to some embodiments, the moment M1 may be related to FFG1, LFG1, T, LB, LF1, and FPR so as to satisfy the following mathematical formula IV:

M2=(FFG1)(0.5)(LFG1)+((T)(cos(θ))+LB+LPR+(LF1)(Tan(θ)))(FPR)(Tan(30))

Formula IV is useful in selecting at least a length LFG1 for the finger-grip element 114 when making (e.g., designing, constructing, manufacturing, etc.) the finger-grip element 114. For example, it may be desirable for the moment M2 in formula IV to be equal to or approximately equal to zero. In at least some embodiments, having M2 be equal to or approximately equal to zero may ensure that the syringe 12 is prevented or at least inhibited from rotating about the distal end 33 of the needle 32 during delivery device. Such stability of the syringe 12 and/or needle 32 may, in at least some embodiments, provide for a relatively comfortable injection for the patient. Thus, according to some embodiments, a method of making the finger-grip element 114 may involve selecting a length LFG1 for the finger-grip element 114 that results in M2 in formula IV being equal to or approximately equal to zero. In scenarios where there is an ability to select dimensions of the syringe 12, the foregoing method may additionally or alternatively involve selecting a length LB of the barrel 20 of the syringe 12 and/or a length LPR of the plunger rod 34 of the syringe 12.

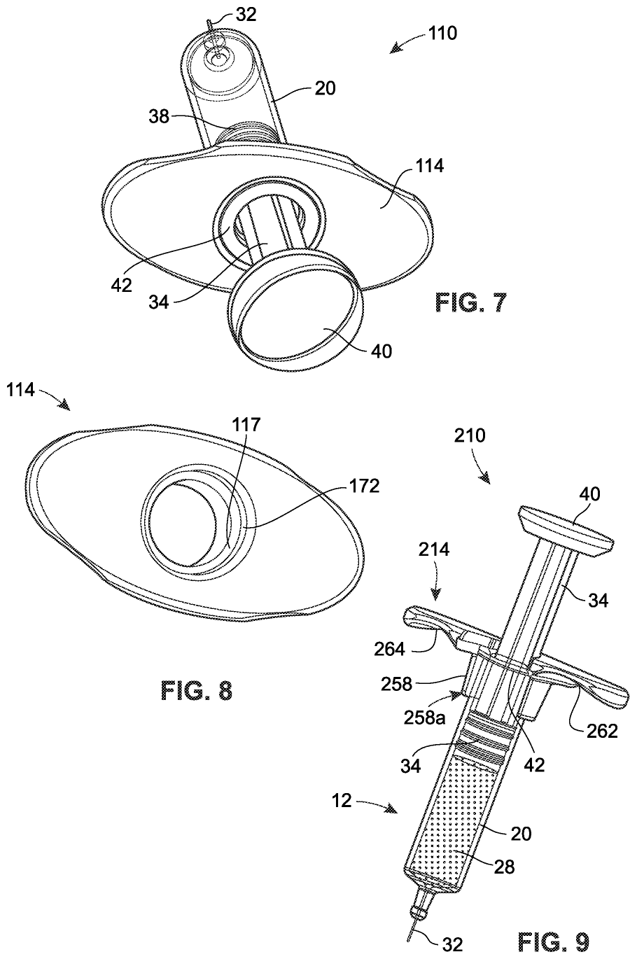

FIG. 7 illustrates another view of the drug delivery device 110 shown in FIG. 6. Here it can be seen that the finger-grip element 114 completely surrounds the barrel 20 of the syringe 12. A proximally facing interior surface 170 (FIG. 8) of the finger-grip element 114 may contact the barrel flange 42, for example, to prevent or inhibit the barrel 20 from moving in the proximal direction with respect to the finger-grip element 114 at least during use of the syringe 12 to deliver the drug. Additionally, in some embodiments, a radially inwardly facing surface 172 (FIG. 8) of the finger-grip element 114 may contact the barrel flange 42 to form a snap-fit coupling, a press-fit coupling, an interference-fit coupling, friction-fit coupling, or a similar coupling with the barrel flange 42, for example, to prevent or inhibit the barrel 20 from moving in the distal direction with respect to the finger-grip element 114.

FIG. 9 illustrates an alternative embodiment of the finger-grip element 114 illustrated in FIGS. 6-8. The finger-grip element 214 is shown as being assembled in combination with the syringe 12 to define a drug delivery device 210. The finger-grip element 214 depicted in FIG. 9 may be structurally and/or functionally similar to the finger-grip element 114 in FIGS. 6-8, except that, for example, the finger-grip element 214 does not completely surround the barrel 20 of the syringe 12. Rather, the finger-grip element 214 (similar to the finger-grip element 14 depicted in FIGS. 1-5) has a side opening 258a formed in the lateral side 258 of the finger-grip element 214 such that the finger-grip element 214 only partially surrounds the barrel 20 of the syringe 12. As described above with respect to the side opening 58a of the finger-grip element 14, the side opening 258a of the finger-grip element 214 may facilitate lateral insertion of the barrel 20 into the finger-grip element 214.

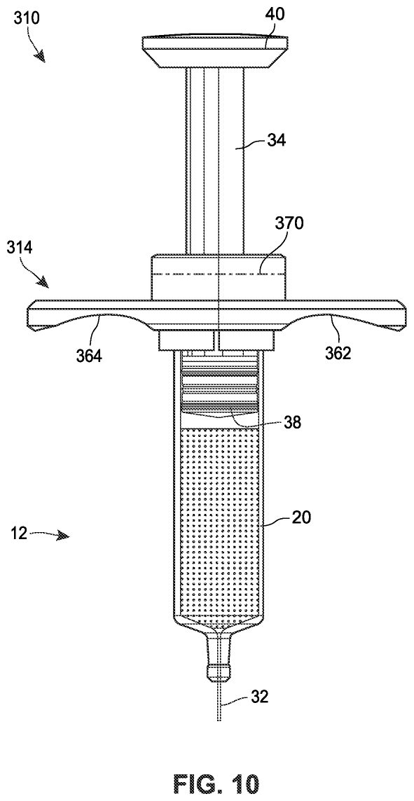

FIG. 10 illustrates another embodiment of a finger-grip element, denoted with reference numeral 314. The finger-grip element 314 is illustrated as being assembled in combination with the syringe 12 to define a drug delivery device 310. The finger-grip element 314 in FIG. 10 may be structurally and/or functionally similar to the finger-grip element 114 in FIGS. 6-8, except for, for example, the axial location of the first and second distally facing exterior surfaces 362 and 364 of the finger-grip 314. More particularly, the first and second distally facing exterior surfaces 362 and 364 of the finger-grip element 314 may closer to the distal end 356 of the finger-grip element 314, as compared to their counterparts in finger-grip element 114. As an example, the first and second distally facing exterior surfaces 362 and 364 may be entirely distal to the proximally facing interior surface 370 (shown in hidden lines in FIG. 10) of the finger-grip element 314 and/or the barrel flange 42 of the syringe 12. The axial distance by which the first and second distally facing exterior surfaces 362 and 364 are offset in the distal direction from proximally facing interior surface 370 of the finger-grip element 314 and/or the barrel flange 42 of the syringe 12 may be selected based on a predetermined or desired pivot location to exist during use of the syringe 12 by the user to expel the drug.

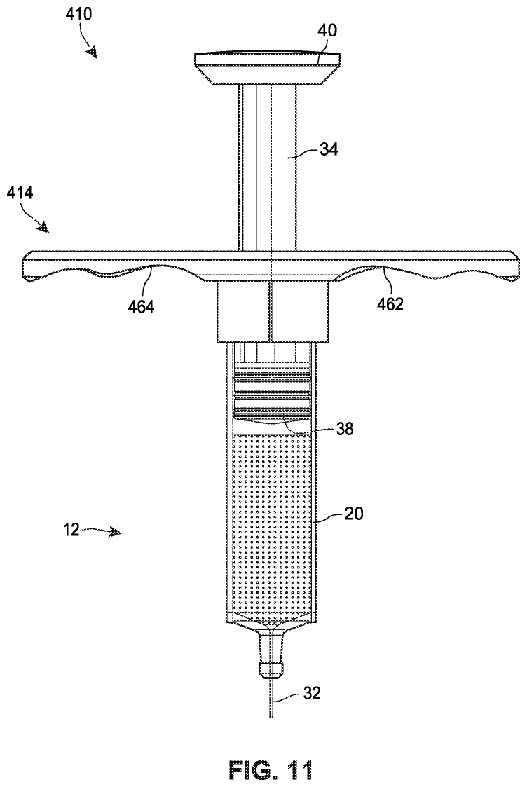

FIG. 11 illustrates another embodiment of a finger-grip element, denoted with reference numeral 414. The finger-grip element 414 is illustrated as being assembled in combination with the syringe 12 to define a drug delivery device 410. The finger-grip element 414 in FIG. 11 may be structurally and/or functionally similar to the finger-grip element 114 in FIGS. 6-8, except for, for example, the first contour of the first distally facing exterior surface 462 and the second contour of the second distally facing exterior surface 464. More particularly, the first contour of the first distally facing exterior surface 462 may include two or more separate finger-grip locations (instead of a single finger-grip location), and the second contour of the first distally facing exterior surface 464 may include two or more separate finger-grip locations (instead of a single finger-grip location). As an example, as shown in FIG. 11, the first contour of the first distally facing exterior surface 462 may include two troughs or depressions arranged adjacent to each other in the radial direction, and the second contour of the second distally facing exterior surface 464 may include two troughs or depressions arranged adjacent to each other in the radial direction. In alternative embodiments, the first and second distally facing exterior surface 462 and 464 may be disposed distal to the position shown in FIG. 11, for example, similar to the location distally facing exterior surfaces of the finger-grip element illustrated in FIG. 10.

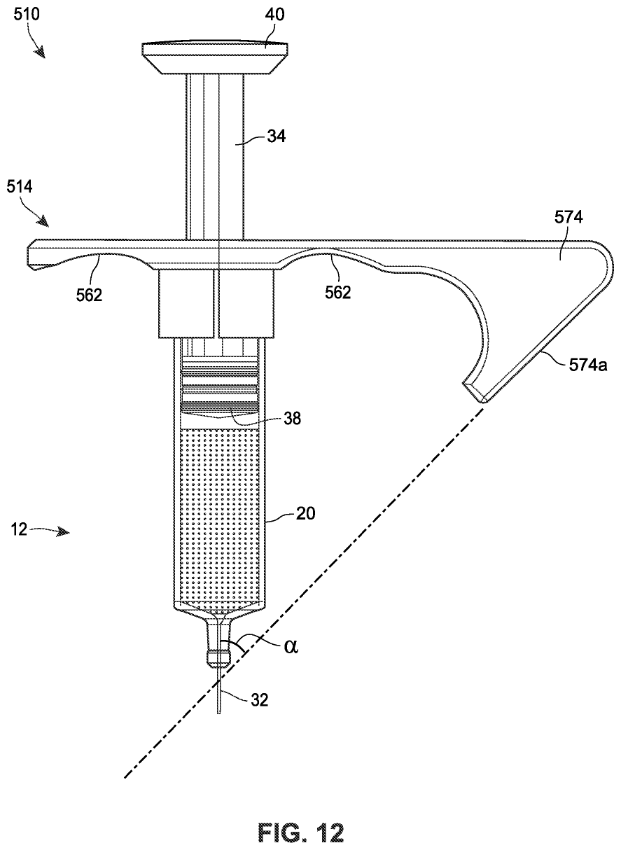

FIG. 12 illustrates another embodiment of a finger-grip element, denoted with reference numeral 514. The finger-grip element 514 is illustrated as being assembled in combination with the syringe 12 to define a drug delivery device 510. The finger-grip element 514 in FIG. 12 may be structurally and/or functionally similar to the finger-grip element 114 in FIGS. 6-8, except for, for example, the inclusion of a stabilizer 574. As an example, the stabilizer 574 may be configured to contact a patient (e.g., the patient's skin) to assist with maintaining the longitudinal axis A of the barrel 20 and/or a longitudinal axis of the needle 32 at a predetermined injection angle α with respect to the patient (e.g., the patient's skin adjacent to an injection site) during use of the syringe 12 to deliver the drug to the patient at the injection site. As a more specific example, the stabilizer 574 may be disposed radially outwardly of the first distally facing exterior surface 562 and may include an outer surface 574a for contacting and/or resting against the patient's skin when the syringe 12 is arranged at the predetermined injection angle α with respect to the patient. As an even more specific example, the outer surface 574a of the stabilizer 574 may be configured such that, when the finger-grip element 514 is coupled with the barrel 20 of the syringe 12, the outer surface 574a is oriented at the predetermined injection angle α with respect to the longitudinal axis A of the barrel 20 and/or a longitudinal axis of the needle 32.

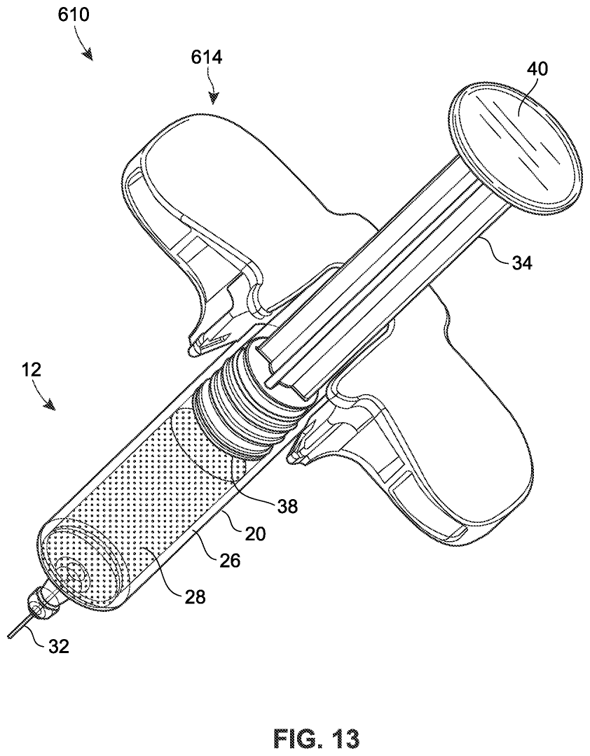

Turning to FIGS. 13-18, illustrated is another embodiment of a drug delivery device 610 in accordance with principles of the present disclosure. The drug delivery device 610 may include the syringe 12 and a finger-grip element 614. Aspects of the syringe 12, including, for example, aspects of the plunger rod 34 and/or the plunger 38, described below in connection with the drug delivery device 610 may apply to any and all of the other embodiments described herein, including, for example, the drug delivery devices 10, 110, 210, 310, 410, and/or 510.

The finger-grip element 614 may be structurally and/or functionally similar to the finger-grip element 14 described above, except for, for example, the configuration of the first distally facing exterior surface 662 and/or the second distally facing exterior surface 664. More particularly, the first distally facing exterior surface 662 may have a first contour that is partially or entirely planar. As an example, the first contour of the first distally facing exterior surface 662 may be partially or entirely perpendicular or substantially perpendicular to the longitudinal axis A. As a more specific example, a radially outer portion of the first distally facing exterior surface 662 may be level or substantially level in the axial direction with a radially inner portion of the first distally facing exterior surface 662. Alternatively, at least a portion of the radially outer portion of the first distally facing exterior surface 662 may be distal to at least a portion of the radially inner portion of the first distally facing exterior surface 662.

Similar to the first distally facing exterior surface 662, the second distally facing exterior surface 664 may have a second contour that is partially or entirely planar. As an example, the second contour of the second distally facing exterior surface 664 may be partially or entirely perpendicular or substantially perpendicular to the longitudinal axis A. As a more specific example, a radially outer portion of the second distally facing exterior surface 664 may be level or substantially level in the axial direction with a radially inner portion of the second distally facing exterior surface 664. Alternatively, at least a portion of the radially outer portion of the first distally facing exterior surface 664 may be distal to at least a portion of the radially inner portion of the first distally facing exterior surface 664.

The distal end 47 of the plunger rod 34 may be coupled with (e.g., removably coupled with) at least the proximal end 49 of the plunger 38. In some embodiments, coupling the plunger rod 34 to the plunger 38 may deform (e.g., elastically deform) a portion of or the entirety of the plunger 38. As an example, coupling the plunger rod 34 to the plunger 38 may cause an outer wall and/or other portion of the plunger 38 to deform such that friction (e.g., sliding friction) between an exterior surface of the outer wall of the plunger 38 and an interior surface of the wall 26 of the barrel 20 during axial movement (e.g., movement in the distal and/or proximal direction) of the plunger 38 with respect to the barrel 20 is reduced, as compared to if the plunger 38 was not deformed by the plunger rod 34. Such deformation of the plunger 38 can be achieved by configuring the plunger rod 34 and/or plunger 38 as described below.

The distal end 47 of the plunger rod 34 may include a threaded exterior surface 80, a plunger rod flange 82, and a distally facing end surface 47a. The plunger rod flange 82 may be proximal to a portion of or the entirety of the threaded exterior surface 80. The plunger rod flange 82 may extend generally radially outwardly with respect to the longitudinal axis A of the barrel 20 and/or a longitudinal axis of the plunger rod 34. The plunger rod flange 82 may have a distally facing surface 82a. The plunger rod 34 may have an axial length L1 corresponding to a distance between the distally facing end surface 47a of the distal end 47 of the plunger rod 34 and the distally facing surface 82a of the plunger rod flange 82 of the plunger rod 34. As an example, the axial length L1 may be equal to or larger than an axial length of the threaded exterior surface 80.

The plunger 38 may include a recess 84 (e.g., a cavity, slot, depression, channel, interior space, etc.) and an axial opening 86. As an example, the axial opening 86 may be formed in a proximally facing exterior surface 38a of the plunger 38 and/or may communicate with (e.g., open into) the recess 84. A distal end of the recess 84 may be closed off by a proximally facing interior surface 38b of the plunger 38. The plunger 38 may have an axial length L2 corresponding to a distance between the proximally facing exterior surface 38a of the plunger 38 and the proximally facing interior surface 38b of the plunger 38. Furthermore, the plunger 38 may include a threaded interior surface 88.

Coupling the plunger rod 34 to the plunger 38 may include engaging (e.g., threadably engaging) the threaded exterior surface 80 of the plunger rod 34 and the threaded interior surface 88 of the plunger 38. As an example, the distal end 47 of the plunger rod 34 may be screwed into the plunger 38. As a more specific example, the distal end 47 of the plunger rod 34 may be inserted in the axial direction through the axial opening 86 in the proximal end 49 of the plunger 38 while rotating the plunger rod 34 with respect to the plunger 38 in order to threadably engage the threaded exterior surface 80 of the plunger rod 34 and the threaded interior surface 88 of the plunger 38. As a more specific example, the distal end 47 of the plunger rod 34 may be threadably advanced in the axial direction into the recess 84 of the plunger 38 at least until the distally facing end surface 47a of the plunger rod 34 comes into contact with the proximally facing interior surface 38b of the plunger 38.

In some embodiments, the axial length L1 of the plunger rod 34 may be larger than the axial length L2 of the plunger 38, at least prior to coupling the plunger rod 34 with the plunger 38. In such embodiments, screwing the plunger rod 34 into the plunger 38 may involve continuing to screw the plunger rod 34 into the plunger 38 after the distally facing end surface 47a of the plunger rod 34 comes into contact with the proximally facing interior surface 38b of the plunger 38. This additional screwing may continue until the distally facing surface 82a of plunger rod flange 82 comes into contact with and/or presses against the proximally facing exterior surface 38a of the proximal end 49 of the plunger 38. As a consequence of this additional screwing, the plunger 38 may deform (e.g., elastically deform) in the axial and/or radial directions to accommodate the distal end 47 of the plunger rod 34 within the recess 84. The plunger rod 34 and/or plunger 38 may be configured (e.g., dimensioned) such that this deformation has an advantageous effect on the performance of the plunger 38 during use of the syringe 12. For example, the deformation of the plunger 38 caused by coupling the plunger 38 to the plunger rod 34 may reduce the friction (e.g., sliding friction) between an exterior surface of the outer wall of the plunger 38 and the interior surface of the wall 26 of the barrel 20 during axial movement (e.g., movement in the distal and/or proximal direction) of the plunger 38 with respect to the barrel 20, as compared to if the outer wall of the plunger 38 was not deformed by the plunger rod 34.

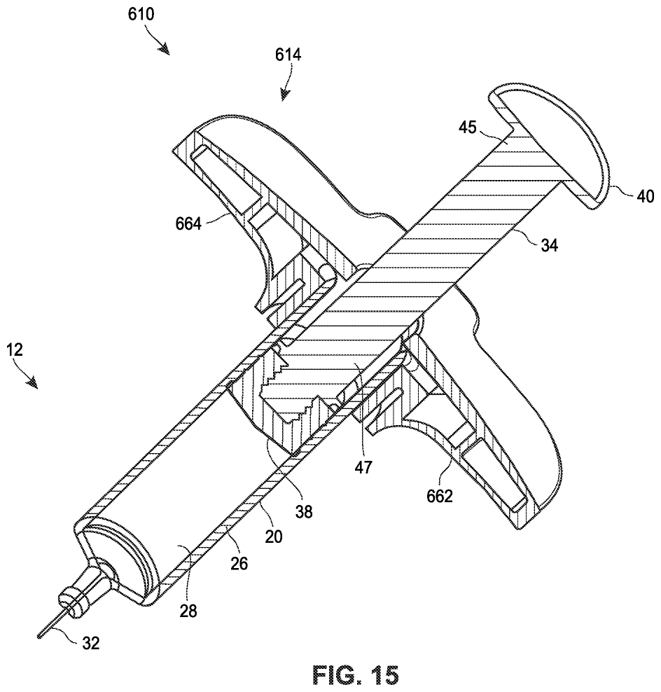

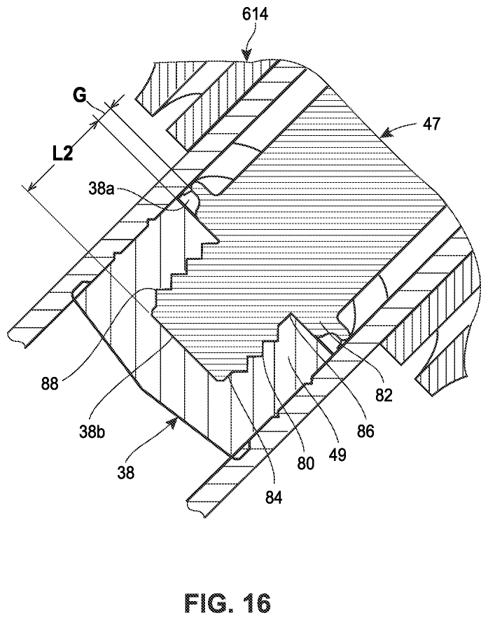

In some embodiments, a limited portion of the distally facing surface 82a of the plunger rod flange 82 may contact and/or press against the proximally facing exterior surface 38a of the plunger 38. For example, the distally facing surface 82a of the plunger rod flange 82 may include a radially inner portion 90 and a radially outer portion 92, wherein the radially inner portion 90 is closer to the longitudinal axis A than the radially outer portion 92. As seen FIGS. 15 and 16, only the radially inner portion 90 of the distally facing surface 82a of plunger rod flange 82 may contact and/or press against the proximally facing exterior surface 38a of the plunger 38, when the plunger rod 34 is screwed into or otherwise coupled with the plunger 38. The radially outer portion 92 of the distally facing surface 82a of plunger rod flange 82 may be spaced from the proximally facing exterior surface 38a of the plunger 38 by a gap G. In having the plunger rod flange 82 contact and/or press against a radially inner portion, but not a radially outer portion, of the proximally facing exterior surface 38a of the plunger 38, the plunger rod flange 82 may cause the plunger 38 to deform (e.g., elastically deform) in a mechanically advantageous manner, including, for example, in a manner that reduces the friction (e.g., sliding friction) between an exterior surface of the outer wall of the plunger 38 and the interior surface of the wall 26 of the barrel 20 during axial movement (e.g., movement in the distal and/or proximal direction) of the plunger 38 with respect to the barrel 20. This, in turn, may reduce an amount of force that the user is required to apply to the finger-grip element 614 and/or plunger rod 34 in order to, for example, expel the drug from the syringe 12 during drug delivery.

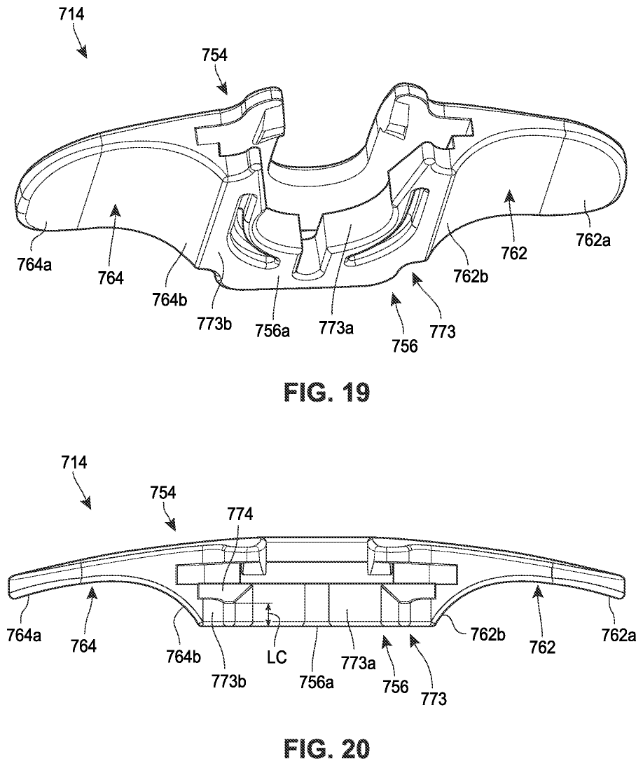

Referring now to FIGS. 19 and 20, illustrated is another embodiment of a finger-grip element 714 in accordance with principles of the present disclosure. The finger-grip element 714 may be used in conjunction with the syringe 12 similar to the other finger-grip element embodiments described herein. The finger-grip element 714 may be structurally and/or functionally similar to the finger-grip element 14 described above, except that, for example, an axial length of a collar of the finger-grip element 714 is less than a corresponding portion of the finger-grip element 14.

More particularly, as seen in FIGS. 19 and 20, the distal end 756 of the finger-grip element 714 may include a collar 773. The collar 773 may be configured to partially surround the barrel 20 of the syringe 12 as seen in FIGS. 19 and 20, or, in alternative embodiments, completely surround the barrel 20 of the syringe 12. At least a portion of the collar 773 may be disposed, in the axial direction, distal to at least a portion of the first distally facing exterior surface 762 and/or at least a portion of the second distally facing exterior surface 764. As an example, a distal-most portion of the collar 773 may be disposed, in the axial direction, distal to a proximal-most portion of the first distally facing exterior surface 762 and/or a proximal-most portion of the second distally facing exterior surface 764. As a more specific example, the distal-most portion of the collar 773 may be disposed, in the axial direction, distal to a distal-most portion of the first distally facing exterior surface 762 and/or a distal-most portion of the second distally facing exterior surface 764. As an even more specific example, the distal-most portion of the collar 773 may be disposed, in the axial direction, distal to the outer portion 762a of the first distally facing exterior surface 762 and/or the outer portion 764a of the second distally facing exterior surface 764.

The collar 773 may have an inner portion 773a configured to contact the barrel 20 of the syringe 12 and an outer portion 773b. As an example, the inner portion 773a of the collar 773 may be closer than the outer portion 773b of the collar 773 to the longitudinal axis A. As a more specific example, the outer portion 773b of the collar 773 may be defined at least in part by the inner portion 762a of the first distally facing exterior surface 762 and/or the inner portion 764a of the second distally facing exterior surface 764.

As seen in FIG. 20, the collar 773 may have a length LC measured in a direction parallel to the longitudinal axis A. As an example, the length LC of the collar 773 may be measured between a lead-in surface 774 for the barrel flange 42 and a distal-most surface 756a of the distal end 756 of the finger-grip element 714. The length LC may be selected such that it is less than: (a) a diameter and/or width of an index finger of an average adult or other user; and/or (b) a diameter and/or width of a middle finger of an average adult or other user. As a consequence of the length LC and/or a reduced wall thickness of the collar 773, a user, during use of the syringe 12 to deliver a drug, may be able to position his or her: (a) index finger concurrently (e.g., simultaneously or substantially simultaneously) in contact with both the finger-grip element 714 and the barrel 20 of the syringe 12; and/or (b) middle finger concurrently in contact with both the finger-grip element 714 and the barrel 20 of the syringe 12. As an example, the index finger may concurrently contact both the first distally facing surface 762 of the finger-grip element 714 and a first lateral side of the barrel 20 of the syringe 12, and the middle finger may concurrently contact both the second distally facing surface 764 of the finger-grip element 714 and a second lateral side of the barrel 20 of the syringe 12. By allowing one or both of the index finger and the middle finger to contact the barrel 20 of the syringe 12, a width of the “V” like gap separating the index finger and the middle finger may be reduced during use of the syringe 12 by the user to deliver the drug. In at least some situations, this results in less twisting or other undesirable torqueing of the user's hand and/or the syringe 12 and/or may be more comfortable and/or ergonomic for the user.

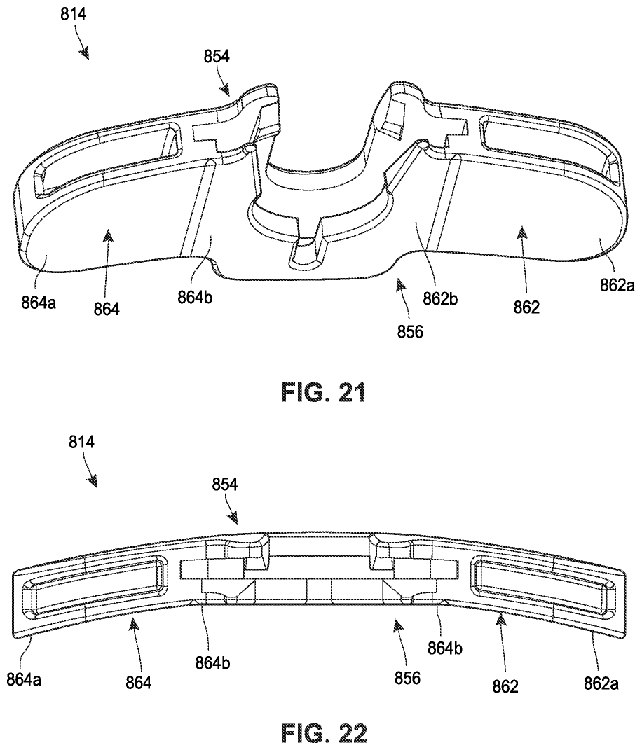

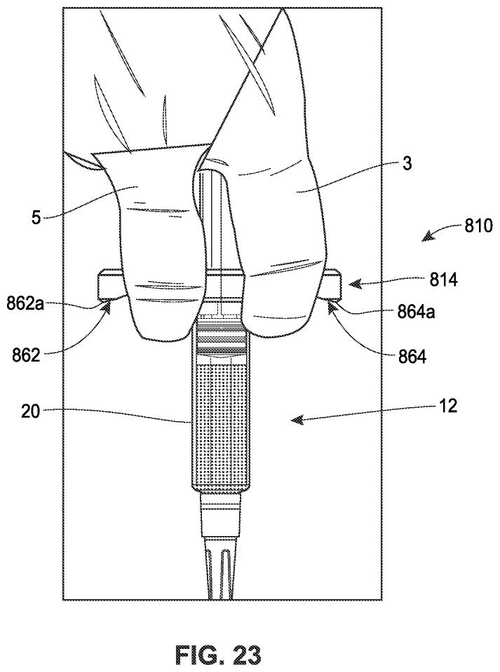

Turning to FIGS. 21-23, illustrated is another embodiment of a finger-grip element 814 in accordance with principles of the present disclosure. The finger-grip element 814 may be used in conjunction with the syringe 12 to define a drug delivery device 810, as seen in FIG. 23. The finger-grip element 814 may be structurally and/or functionally similar to the finger-grip element 714 described above, except that, for example, the finger-grip element 814 omits any element equivalent to the collar 773.

Due to the lack of a collar element, the inner portion 862b of the first distally facing exterior surface 862 of the finger-grip element 814 may define the proximal-most portion of the first distally facing exterior surface 862 and/or the inner portion 864b of the second distally facing exterior surface 864 of the finger-grip element 814 may define the proximal-most portion of the second distally facing exterior surface 864, as seen in FIG. 22. Furthermore, as seen in FIG. 22, the outer portion 862a of the first distally facing exterior surface 862 may be disposed, in the axial direction, distal to at least a portion of or the entirety of the inner portion 862b of the first distally facing exterior surface 862; and/or the outer portion 864a of the second distally facing exterior surface 864 may be disposed, in the axial direction, distal to at least a portion of or the entirety of the inner portion 864b of the second distally facing exterior surface 864.

FIG. 23 illustrates the finger-grip element 814 being held by person's hand as would be done during use of the syringe 10 to deliver a drug. As seen in FIG. 23, the configuration of the finger-grip element 814 allows the user's index finger 3 and middle finger 5 to contact an exterior surface of the barrel 20 of the syringe 12. Thus, the width of the “V” like gap separating the index finger 3 and the middle finger 5 is reduced (as compared to if the user's index finger 3 and/or middle finger 5 were separated from the exterior surface of the barrel 20 by a portion of a finger-grip element). In at least some situations, this results in less twisting or other undesirable torqueing of the user's hand and/or the syringe 12 during use of the syringe 12 to deliver the drug and/or may be more comfortable and/or ergonomic for the user. Furthermore, as seen in FIG. 23, the user during use of the syringe 12 to deliver a drug may be able to position his or her: (a) index finger 3 concurrently (e.g., simultaneously or substantially simultaneously) in contact with both the second distally facing exterior surface 864 of the finger-grip element 814 and the exterior surface of a second lateral side of the barrel 20 of the syringe 12; and/or (b) middle finger 3 concurrently in contact with both the first distally facing exterior surface 862 of the finger-grip element 814 and the exterior surface of a first lateral side of the barrel 20 of the syringe 12. Moreover, as seen in FIG. 23, at least a first portion of the exterior surface of the first lateral side of the barrel 20 of the syringe 12 may be disposed, in the axial direction, proximal to the outer portion 862a of the first distally facing exterior surface 862 of the finger-grip element 814 and distal to the inner portion 862b of the first distally facing exterior surface 862 of the finger-grip element 814; and/or at least a second portion of the exterior surface of the second lateral side of the barrel 20 of the syringe may be disposed, in the axial direction, proximal to the outer portion 864a of the second distally facing exterior surface 864 of the finger-grip element 814 and distal to the inner portion 864b of the second distally facing exterior surface 864 of the finger-grip element 814. Moreover, as seen in FIG. 23, the at least a first portion of the exterior surface of the first lateral side of the barrel 20 of the syringe 12 may be free from contact with the finger grip element 814 and/or not covered by the finger grip element 814; and/or the at least a second portion of the exterior surface of the second lateral side of the barrel 20 of the syringe 12 may be free from contact with the finger grip element 814 and/or not covered by the finger grip element 814.

All features described herein, including in the specification, claims, abstract, and drawings, and all the steps in any method or process described herein, may be combined in any combination, except combinations where one or more of the features and/or steps are mutually exclusive.

As will be recognized, the devices and methods according to the present disclosure may have one or more advantages relative to conventional technology, any one or more of which may be present in a particular embodiment in accordance with the features of the present disclosure included in that embodiment. Other advantages not specifically listed herein may also be recognized as well.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP lib/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(18)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1-27. (canceled)

28. A finger-grip element for use with a syringe, the finger-grip element comprising:
a proximal end and a distal end disposed at respective axial positions along a longitudinal axis;
an opening configured to receive at least portion of the syringe; and
a first distally facing surface comprising a first contour configured to:
(a) inhibit or prevent at least a first finger of a user from moving in a radially outward direction with respect to the longitudinal axis during use of the syringe to deliver a drug, and/or
(b) exert a first reaction force on at least the first finger of the user in a direction parallel or substantially parallel to the longitudinal axis during use of the syringe to deliver the drug.

29. The finger-grip element of claim 28, wherein the first distally facing surface comprises an outer portion and an inner portion, the inner portion being closer than the outer portion to the longitudinal axis, at least a portion of the outer portion being distal to at least a portion of the inner portion.

30. The finger-grip element of claim 29, wherein the outer portion of the first distally facing surface of the finger-grip element is configured to inhibit or prevent at least the first finger of the user from moving in the radially outward direction with respect to the longitudinal axis during use of the syringe to deliver the drug.

31. The finger-grip element of claim 28, wherein the finger-grip element comprises a second distally facing surface having a second contour configured to:
(a) inhibit or prevent at least a second finger of a user from moving in a radially outward direction with respect to the longitudinal axis during use of the syringe to deliver the drug, and/or
(b) exert a second reaction force on at least the second finger of the user in a direction parallel or substantially parallel to the longitudinal axis during use of the syringe to deliver the drug, and wherein
the first distally facing surface and the second distally facing surface are optionally disposed on opposite sides of the longitudinal axis.

32. (canceled)

33. The finger-grip element of claim 31, wherein the first contour and the second contour are symmetric with each other across the longitudinal axis.

34. A method of making a finger-grip element for a syringe, the finger-grip element having a length LFG1 corresponding to a distance between a longitudinal axis of a barrel of the syringe and a distally facing surface of the finger-grip element for contacting a finger of a user during use of the syringe to deliver a drug, the method comprising:
selecting the length LFG1 for the finger-grip element such that M is equal to or approximately equal to zero in a mathematical formula wherein: M=(FFG1)(0.5)(LFG1)+((T)(cos(θ))+LB+LPR+(LF1)(Tan(θ)))(FPR)(Tan(30)); and
wherein:
FFG1 corresponds to a force applied to the distally directed surface of the finger-grip element by the finger of the user during use of the syringe to deliver the drug,
T corresponds to a distance between a wrist of the user and a proximal end of a plunger rod of the syringe,
LB corresponds to a length of barrel of the syringe,
LPR corresponds to a length of the plunger rod of the syringe,
LF1 corresponds to a length of the finger of the user,
θ corresponds to an angle between the finger of the user and a thumb of the user during use of the syringe to deliver the drug, and
FPR corresponds to a force applied to the plunger rod by the thumb of the user during use of the syringe to deliver the drug.

35. The method of claim 34, wherein M corresponds to a moment at or near a distal end of a needle of the syringe.

36. The method of claim 34, wherein the finger of the user corresponds to an index finger of the user.

37. The method of claim 34, wherein the force FPR is applied to the plunger rod in a direction parallel to and/or coaxial with the longitudinal axis of the barrel.

38. The method of claim 34, wherein the distally facing surface of the finger-grip element has a contour configured to:
(a) inhibit or prevent at least the finger of a user from moving in a radially outward direction with respect to the longitudinal axis during use of the syringe to deliver the drug, and/or
(b) exert a reaction force on at least the finger of the user in a direction parallel to or substantially parallel the longitudinal axis during use of the syringe to deliver the drug; and
wherein at least a portion of the contour of the distally facing surface of the finger-grip element optionally has a concave curvature.

39. (canceled)

40. The method of claim 34, wherein the distally facing surface of the finger-grip element comprises an outer portion and an inner portion, the inner portion being closer than the outer portion to the longitudinal axis, at least a portion of the outer portion being distal to at least a portion of the inner portion.

41. The method of claim 40, wherein the outer portion of the distally facing surface of the finger-grip element is configured to inhibit or prevent at least the finger of the user from moving in a radially outward direction with respect to the longitudinal axis during use of the syringe to deliver the drug.

42. The method of claim 34, wherein the finger-grip element is configured to form a snap-fit coupling with the barrel of the syringe.

43. A drug delivery device comprising:
a syringe comprising:
a barrel including a proximal end, a distal end, and a longitudinal axis,
a barrel flange disposed at the proximal end of the barrel and extending radially outwardly with respect to the longitudinal axis, and
a plunger at least partially disposed within the barrel and moveable along the longitudinal axis;
a finger-grip element coupled with the barrel flange and comprising a distally facing surface; and
wherein, during use of the syringe to deliver a drug, the finger-grip element is configured to allow at least one finger of a user to contact concurrently the distally facing surface of the finger-grip element and the barrel of the syringe.

44. The drug delivery device of claim 43, wherein the distally facing surface of the finger-grip element comprises an inner portion and an outer portion, the inner portion being closer than the outer portion to the longitudinal axis of the barrel.

45. The drug delivery device of claim 44, wherein at least a portion of the outer portion of the distally facing surface of the finger-grip element is distal to at least a portion of the inner portion of the distally facing surface of the finger-grip element.

46. The drug delivery device of claim 44, wherein the outer portion of the distally facing surface of the finger-grip element (a) defines a distal-most portion of the finger-grip element, and/or (b) is configured to inhibit or prevent the at least one finger of the user from moving in a radially outward direction with respect to the longitudinal axis during use of the syringe to deliver the drug.

47. (canceled)

48. The drug delivery device of claim 44, wherein at least a portion of an exterior surface of the barrel is proximal to the outer portion of the distally facing surface of the finger-grip element and distal to the inner portion of the distally facing surface finger-grip element.

49. The drug delivery device of claim 48, wherein the at least portion of the exterior of surface of the barrel is free from contact with the finger-grip element and/or not covered by the finger-grip element.

50. The drug delivery device of claim 43, wherein the distally facing surface comprises a first contour configured to: (a) inhibit or prevent the at least one finger of the user from moving in a radially outward direction with respect to the longitudinal axis during use of the syringe to deliver the drug, and/or (b) exert a first reaction force on the at least one finger of the user in a direction parallel or substantially parallel to the longitudinal axis during use of the syringe to deliver the drug.
